(19) United States
(12) Patent Application Publication
(10) Pub. No.: US 2026/0179645 A1
(43) Pub. Date: Jun. 25, 2026

(54) HEARING DIAGNOSTIC METHOD AND SYSTEM EMPLOYING ARTIFICIAL INTELLIGENCE

(71) Applicant: Instytut Fizjologii i Patologii Sluchu, Nadarzyn (PL)

(72) Inventors: Andrzej Czyzewski, Warszawa (PL); Henryk Skarzynski, Warszawa (PL); Bozena Kostek, Warszawa (PL); Piotr H. Skarzynski, Warszawa (PL)

(21) Appl. No.: 19/416,496

(22) Filed: Dec. 11, 2025

(30) Foreign Application Priority Data
Dec. 20, 2024 (PL) 24460040.9

(51) Int. Cl. G10L 25/66; G10L 21/0208; G10L 25/30; G16H 10/20; G16H 50/20

(52) U.S. Cl. CPC G10L 25/66; G10L 21/0208; G10L 25/30; G16H 10/20; G16H 50/20

(57) ABSTRACT

A system and method for diagnosing hearing impairments in individuals, particularly school-aged children, using voice sample analysis and data from electronic questionnaires. The system utilizes a deep neural network to process extracted acoustic features from voice recordings and correlate them with hearing performance data, generating a predicted audiogram. The system includes a microphone, signal acquisition and processing blocks, a neural network-based analysis engine, and a result interpretation interface.

### CROSS-REFERENCES TO RELATED APPLICATIONS

This application claims the benefit of E.P. Patent Application No. 24460040.9, filed Dec. 11, 2024, the entire contents of which are incorporated by reference.

### FIELD OF THE INVENTION

The present invention relates to diagnostic systems for evaluating auditory perception, and more particularly, to a method and system for performing hearing diagnostics using voice sample analysis combined with electronic questionnaire data, processed via artificial intelligence.

### BACKGROUND

There are well-known and widely used methods of hearing testing based on screening, which provide only general information about the state of hearing, indicating whether it deviates from the normative.

Currently, hearing diagnosis is based mainly on audiometric testing, which requires specialized equipment and the presence of qualified medical personnel. These methods are time-consuming, expensive, and not always accessible, especially in remote or underserved areas with limited medical care.

A need therefore exists for a solution that overcomes these limitations.

In US Patent Application Publication No. 20020107692A1, a method and system are proposed for rapid and reliable testing of speech intelligibility in children. This invention presents a system for assessing children's ability to understand speech under various acoustic conditions.

A method for training a deep neural network for acoustic modeling in speech recognition is described in U.S. Pat. No. 9,842,610B2. The solution, according to the invention, focuses on the use of deep neural networks for analyzing speech sounds.

In U.S. Pat. No. 9,786,270B2, a method for generating acoustic models that can be utilized in various applications, including speech recognition and sound analysis, is described.

### SUMMARY OF THE INVENTION

One embodiment of the invention is a method of hearing diagnostics for a subject. The method includes receiving a voice sample of the subject speaking a predetermined text; extracting acoustic features from the obtained voice sample; processing the extracted acoustic features by a trained deep neural network; and providing based on the processing a audiogram comprising estimated hearing thresholds at a plurality of frequencies or a hearing impairment diagnosis. The audiogram may be pure tone. The voice sample may be obtained directly from the subject by providing a predetermined text to the subject, prompting the subject to read the text, and recording the sample. Alternatively, a third party such as a nurse could record the voice sample and send a recording of it for analysis.

The method may also include receiving electronic questionnaire data relating to hearing performance about the subject. The questionnaire data may be obtained directly from the subject by providing a questionnaire to the subject and having the questionnaire completed by the subject or with the assistance of a third party such as a nurse on a computer.

Another embodiment of the invention is hearing diagnostic system. The system has a microphone or other acoustic transducer and a signal acquisition device connected to the microphone or other acoustic transducer. The signal acquisition device is for recording a voice sample of the subject speaking a predetermined text and has a first processor. The signal acquisition device may be a laptop or desktop computer, tablet or smartphone which is located close to the subject and may even be operated by the subject.

The system also has a signal processing device comprising a second processor programmed to extract acoustic features from the voice sample and a deep neural network. The deep neural network is for interpreting electronic questionnaire data about the subject and the acoustic features to provide an audiogram comprising estimated hearing thresholds at a plurality of frequencies or a hearing impairment diagnosis. The signal processing device is electronically connected to the signal acquisition device to receive the voice sample from the signal acquisition device. The signal processing device may be a computer or a computer system, which is located remotely from the signal acquisition device.

### BRIEF DESCRIPTION OF THE DRAWINGS

FIG. 1 is a flowchart illustrating the diagnostic process of the invention.

FIG. 2 is a schematic block diagram of the hearing diagnostic system architecture of the invention.

FIG. 3 shows a block diagram integrating portions of the diagnostic process of FIG. 1 and the system of FIG. 2.

FIG. 4 displays the usage interface of the system of FIG. 2, showing the test results and predicted audiogram.

FIG. 5 shows integrated elements of the diagnostic process of FIG. 1 and the system of FIG. 2.

### DETAILED DESCRIPTION OF THE INVENTION

The subject of the invention is a method and system for hearing diagnosis based on analysis of voice samples and data from electronic questionnaires of test subjects, especially school-aged children.

Various solutions for sound analysis and hearing diagnostics are known, which may be relevant to the present invention, but do not impinge on the originality of the proposed system solution.

To develop the method and diagnostic system according to the invention, the results of a well-known neuropsychological test, which involves listening to speech in the presence of noise, were utilized. This test is based on CCITT (Consultative Committee for International Telephony and Telegraphy, aka, ITU-T) standards for evaluating various processes related to attention and executive functions. In this case, this test was used to test the hearing of children and adolescents aged 8 to 17.

It has been established, based on several seconds of voice samples, including in noise, that there are possibilities concerning the creation of hearing characteristics of the tested person, advantageously in the form of an audiogram.

The results of the tests allowed the development and collection of a set of electronic questionnaires from individuals.

The various elements of the invention were developed based on the existing state of the art, particularly in the field of artificial intelligence.

Machine learning is known to utilize DSP signal processors, FPGAs, and ASICs to accelerate and optimize data processing calculations, particularly in applications that require high computational power and low latency, such as real-time signal analysis, image processing, speech recognition, and autonomous control systems. These specialized hardware chips are used to implement machine learning models, enabling advanced parallel computing, which contributes to higher performance and lower power consumption compared to standard general-purpose processors.

It is known from life observations that the way a person hears affects the way they articulate speech. What is also known is that information about auditory perception is contained in voice samples. As a result of the development work, it was found that information about the subject's auditory perception can be extracted using advanced machine learning models, advantageously realized in the form of a deep neural network.

The hearing diagnostic method according to the invention is characterized by the fact that, using a microphone or other acoustic transducer, samples of the subject's voice are taken and recorded on the sound card of the signal acquisition block, after which the sound is converted into digital form. Using a signal processing device, acoustic features are extracted from the recorded voice sample. Following this, in the analysis block, the extracted acoustic features are processed using a deep neural network, along with the data contained in the electronic questionnaires, by the principles of machine learning. As a result, an audiogram is generated and then presented to the user or operator through an interface.

In a variation of the invention, in addition to recording a voice sample, a recording of previously articulated phrases and/or sounds by the test person is also made.

The hearing diagnostic system according to the invention is characterized by the fact that it consists of a microphone or other acoustic transducer, which is connected to a signal acquisition block equipped with a sound card, whereby the signal acquisition block is connected to a signal processing device, which is connected to an analysis block containing a deep neural network designed for training, based also on data from an electronic questionnaire, formed in the data acquisition block. In contrast, the analysis block is connected in turn to a results interpretation block, a user interface, and a measurement display.

The benefit of using the invention lies in the evaluation of hearing in school children and adolescents through acoustic analysis of voice samples using deep neural networks.

The result of the invention is the creation of an effective diagnostic tool that enables the rapid and non-invasive detection of hearing disorders. The solution significantly enhances the diagnosis process, particularly in areas with limited access to specialized medical care.

The invention has the potential to benefit not only the medical sphere but also the social sphere, helping to improve the quality of life for children and adolescents with hearing impairments through the earlier initiation of therapeutic interventions. The invention aligns with the priorities of institutions funding medical research, education, and health insurance, combining advanced technology with genuine social needs.

The invention is explained in more detail in the figures, which illustrate various aspects of the diagnostic system and method.

### Example

The technical implementation of the invention requires the creation of an extensive annotated training data set, in the form of completed electronic questionnaires containing data of at least about 1,000 students. The annotations relate to normal hearing or to diagnosed hearing conditions, such as moderately severe to severe or sloping sensorineural hearing loss.

Beneficially, through a website, voice samples are collected from a large population of students who simultaneously undergo standard audiometric hearing tests to generate an audiogram, which are annotated by hearing specialists based on the audiometric hearings tests, and complete the questionnaire themselves or with the help of a caregiver, such as a parent, teacher, or medical staff. Annotated audiograms are considered the gold standard. The voice samples may be processed in a signal processing device 4 and acoustic features extracted therefrom by a feature extraction module 3 as discussed below. The collected data and extracted features are used to train a deep neural network capable to correlate them with the results of hearing tests and to generate an audiogram.

A sample questionnaire for assessing the hearing of school children and adolescents for use at the stage of collecting material for teaching a machine model based on artificial intelligence is set forth below.

A. General Information about the Students:
1. Student's name:
2. Age of the student:
3. Gender of the student:

### B. Questions Regarding Medical History:

1. Has the student previously had hearing tests? If so, when and what were the results?
2. Has the student had ear infections in the past? Possible answers: never, rarely, and often.
3. Has the student ever complained of ear pain? Possible answers: never, rarely, and often.
4. Has the student had speech problems or speech delays? Possible answers: yes and no.
   C. Questions about Everyday Situations:
1. Does the student struggle to understand speech in noisy environments? Possible answers: never, rarely, and often.
2. Does the student often ask you to repeat what was said? Possible answers: never, rarely, and often.
3. Does the student have difficulty hearing the teacher in class, especially when the teacher is speaking from a greater distance? Possible answers: never, rarely, and often.
4. Does the student adjust the volume of the TV or other devices to hear better? Possible answers: never, rarely, and often.
5. Does the student respond to quiet sounds, such as whispering or the sound of footsteps? Possible answers: always, often, rarely, and never.

### D. Social Behavior Questions:

1. Does the student seem withdrawn or shy in situations where other children are talking? Possible answers: never, rarely, and often.
2. Does the student avoid places with noise, such as the playground or cafeteria? Possible answers: never, rarely, and often.

### E. Sound Response Questions:

1. Does the student hear sounds that seem normal to others, such as the sound of a bell, siren, or clapping? Possible answers: always, often, rarely, and never.
2. Does the student have difficulty distinguishing the direction from which the sound comes? Possible answers: never, rarely, and often.
   F. Type and Severity of Hearing Problems (if any):
1. Does the student have hearing problems in only one ear? Possible answers: yes (right/left) and no.
2. Does the student experience tinnitus (Tinnitus)? Possible answers: never, rarely, and often.
3. Are hearing problems worse at certain times of the day or in certain situations? Possible answers: yes and no.

### G. Other Questions:

1. Is there a history of hearing problems in the student's family? Possible answers: yes and no.
2. Has the student been exposed to deafening sounds or noise for an extended period (e.g., concerts, loud computer games, machines)? Possible answers: yes and no.
   H. Questions about Speaking and Reading
1. Does the student have problems with reading, for example, is he dyslexic? Possible answers: yes and no.
2. Does the student have pronunciation problems, for example, is he a stutterer, or does he articulate incorrectly? Possible answers: yes and no.

Ideally, the same or substantially similar questionnaire is used in the method of diagnosis, not just in the training.

Voice samples for the training data set are created by having a student read the following exemplary text into a microphone for recording in an audio file (preferably, an uncompressed audio file format):
“Hi, my name is [child's name]. Today I will tell you a short story.

On a warm summer day, I decided to go for a walk in the park. The sun shone brightly in the sky, and a gentle wind swayed the leaves on the trees. On the way, I met a small dog that barked happily when it saw children playing. Colorful flowers bloomed in the park, and birds sang their melodies. At one point, I heard a frog retching loudly by the pond. I thought/thought that maybe it wanted to say hello.

I sat down on a bench and watched/watched the ducks swim across the water. Time passed, and the sky slowly changed color. It became cooler, and I noticed that evening was approaching. I regretfully got up and decided to go home. But it was a wonderful day, full of sounds and colors.”

Commentary: the above text contains a variety of sounds that the child will have to pronounce, which includes a wide range of acoustic frequencies, from high (e.g. “ś”, “s”, “z”) to low (e.g. “r”, “ł”, “ż”). There are also sounds associated with the articulation of different types of vowels and consonants, which should support the process of training the audiogram recognition model. Ideally the above text is also used for diagnosing students by the method and system and system of the invention, but other voice samples may be used instead or additionally.

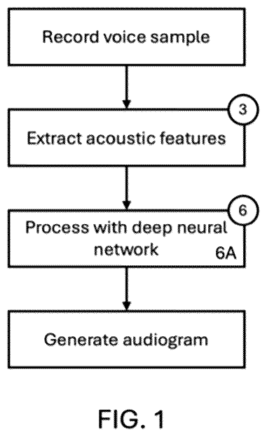

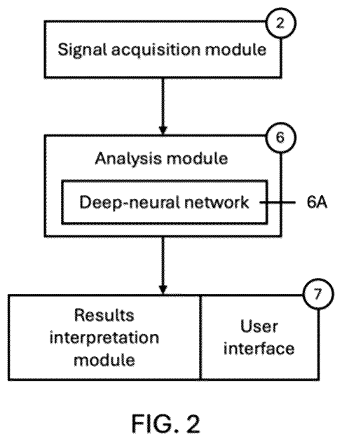

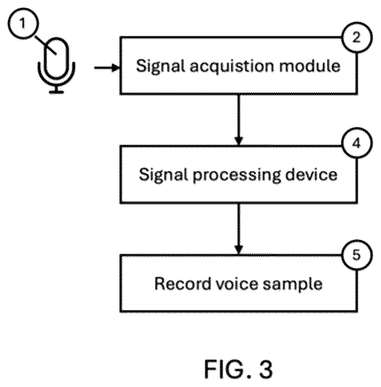

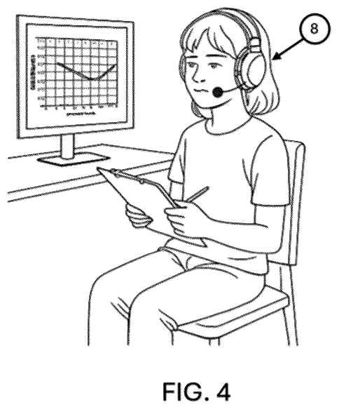

As shown in FIGS. 1-5, the hearing diagnostic system includes components such as microphones, signal acquisition and processing blocks, analysis modules, and user interfaces.

A voice sample is obtained by having the test subject speak specific verbal phrases, typically lasting several seconds, into voice sensor 1, typically a microphone, which creates an analog audio signal. A signal acquisition device 2 converts the analog audio signal to digital form and records the voice sample. Signal acquisition device 2 may include voice sensor 1. Signal acquisition device 2 has a processor programmed to convert the analog audio signal to digital form and record the digitized voice sample. Signal acquisition device 2 may be a smartphone or a computer including a general purpose one.

The digitized voice sample may be further processed in a signal processing device 4 and recorded. Signal processing device 4 may be configured to normalize the digitized voice sample to account for variables such as age, gender, and native language.

An acoustic feature extraction module 3 performs acoustic analysis of the recorded sample, advantageously extracting features such as signal energy, speech rate, formant frequencies, power spectrum, amplitude, and frequency modulations, and cepstral features, preferably the extracted features include power density spectrum and mel-cepstral coefficients. Advantageously, module 3 conducts noise filtering before feature extraction, which removes unwanted noise and interference from the signal, speech, as well as normalization, which unifies the volume level of the signal.

The extracted acoustic features from feature extraction module 3 is an input into an analysis module 6. Another input is the data from an electronic questionnaire regarding the test subject's medical history, hearing, social behavior, speaking and reading ability. The questionnaire is typically similar to the one previously described. A computer having a data acquisition module 5 is used to obtain this data, i.e., the computer is programmed to acquire this data. It is contemplate that the test subject or a caregiver sits at the computer and completes the questionnaire.

The questionnaire data and extracted acoustic features are the primary inputs into analysis module 6. Analysis module 6 has a deep neural network 6A, which based on its training, predicts audiogram 16 of the test subject and provides a diagnosis. Audiogram 16 may be limited to predicted hearing thresholds in dB HL at 125, 250, 500, 1000, 2000, 4000, and 8000 Hz, for example. Neural network 6A is an advanced machine learning model implemented in the form of software for general purpose microprocessor or, advantageously, a hardware chip of a known DSP processor, or known and appropriately programmed FPGA or ASIC.

The results of the hearing screening and predicted audiogram 16 are displayed on the user interface 7, which may include a monitor of a computer, allowing the specialist or caregiver of the person being tested to interpret the results. At the same time, results interpretation module 10 compares predicted audiogram 16 with the norms for the corresponding age and suggests a diagnosis, which is predicted by neural network 6A, and suggests further diagnostic or therapeutic steps. Where the test subject does not suffer from hearing impairment, the diagnosis may be no hearing impairment. Predicted audiogram 16 shows the child's hearing characteristics as a function of frequency. User interface 7 may also allow for entry of answers to the questionnaire and control the obtaining of the voice samples.

An optional storage device stores the collected data, models, and performance history. Voice samples and analysis results are also stored for future reference, enabling continuous model improvement through learning from new data.

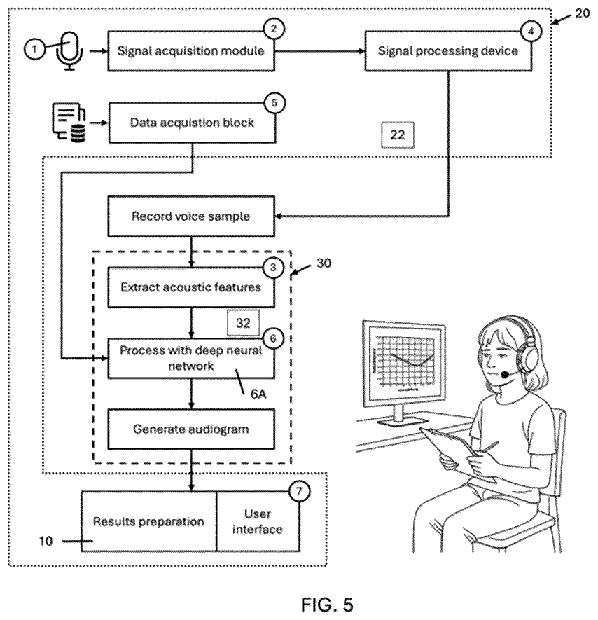

In one embodiment of the system, as best illustrated in FIG. 5, there is a first computer 20. Computer 20 comprises signal acquisition device 2, a first computer processor 22, voice sensor 1, data acquisition module 5 in the form of programming for processor 22, user interface 7 and results interpretation module 10 in the form of programming for processor 22. First computer 20 is connected electronically, typically by a local area network or a wide area network such as the internet, to a second computer 30 having a second computer processor 32. Second computer 30 also has analysis module 6 including deep neural network 6A. Optionally, deep neural network 6A may operate on a third processor. Acoustic feature extraction module 3 may take the form of programming for processor 22 or 32, but preferably for processor 32.

In another embodiment of the system, first computer 20 comprises signal acquisition device 2, a first computer processor 22, voice sensor 1, data acquisition module 5 in the form of programming for first processor 22, user interface 7, results interpretation module 10 in the form of programming for processor 22, acoustic feature extraction module 3 in the form of programming for processor 22, analysis module 6 in the form of programming for first processor 22. In this embodiment, the first processor and the second processor are the same.

While the invention has been described with respect to certain embodiments, as will be appreciated by those skilled in the art, it is to be understood that the invention is capable of numerous changes, modifications and rearrangements, and such changes, modifications and rearrangements are intended to be covered by the following claims.

What is claimed is:

1. A method of hearing diagnostics for a subject comprising:
receiving a voice sample of the subject speaking a predetermined text;
extracting acoustic features from the obtained voice sample;
processing the extracted acoustic features by a trained deep neural network; and
providing based on the processing an audiogram comprising estimated hearing thresholds at a plurality of frequencies or a hearing impairment diagnosis.

2. The method according to claim 1 further comprising receiving electronic questionnaire data relating to hearing performance about the subject.

3. The method according to claim 2, wherein the deep neural network is trained on a dataset comprising voice samples annotated with corresponding audiometric test results and electronic questionnaire responses collected from a population of at least 1,000 school-aged children.

4. The method according to claim 1, wherein the extracted acoustic features include one or more of the following: signal energy, speech rate, formant frequencies, power spectrum, amplitude modulation, frequency modulation, and cepstral coefficients.

5. The method according to claim 1, further comprising noise filtering before the acoustic feature extraction to suppress background noise before feature extraction.

6. The method according to claim 1, wherein the deep neural network is implemented on a hardware accelerator selected from the group consisting of a digital signal processor (DSP), field-programmable gate array (FPGA), or application-specific integrated circuit (ASIC).

7. The method according to claim 1 further comprising comparing the predicted audiogram against age-specific normative thresholds and generating alerts or recommendations for further medical evaluation.

8. The method according to claim 1, wherein multiple voice samples are recorded under different acoustic conditions, including quiet and noisy environments, to enhance the accuracy of the audiogram prediction.

9. The method according to claim 1 further comprising generating a confidence score indicating the confidence level of the audiogram or the hearing impairment diagnosis.

10. The method according to claim 1, wherein the signal processing device includes a calibration module configured to adapt the audiogram prediction model to individual speech characteristics based on demographic variables such as age, gender, and native language.

11. The method according to claim 1, wherein the user interface provides real-time visual or auditory cues to guide the subject during the voice recording process, ensuring optimal data quality.

12. A hearing diagnostic system for a subject comprising:
a microphone or other acoustic transducer;
a signal acquisition device connected to the microphone or other acoustic transducer for recording a voice sample of the subject speaking a predetermined text, the device having a first processor; and
a signal processing device comprising a second processor programmed to extract acoustic features from the voice sample and a deep neural network for interpreting electronic questionnaire data about the subject and the acoustic features to provide an audiogram comprising estimated hearing thresholds at a plurality of frequencies or a hearing impairment diagnosis, the signal processing device electronically connected to the signal acquisition device to receive the voice sample.

13. The system according to claim 12 wherein the signal acquisition device further comprises a user interface configured to allow manual input or correction of questionnaire data and provide interactive feedback to caregivers or healthcare professionals.

14. The system according to claim 12, further comprising data storage configured to retain voice recordings, questionnaire data, and audiogram predictions for continuous learning and performance improvement of the neural network.

15. The system according to claim 12, wherein the signal acquisition device is a mobile device selected from a smartphone and a tablet.

16. The system according to claim 12, wherein the signal processing device is programmed to perform time-frequency analysis using short-time Fourier transform or wavelet transform for more accurate extraction of acoustic features.
